(19) United States
(12) Patent Application Publication
(10) Pub. No.: US 2017/0315194 A1
(43) Pub. Date: Nov. 2, 2017

(54) OPERATING METHOD FOR A MEDICAL IMAGING APPARATUS

(71) Applicant: Siemens Healthcare GmbH, Erlangen (DE)

(72) Inventor: Stefan Schor, Erlangen (DE)

(73) Assignee: Siemens Healthcare GmbH, Erlangen (DE)

(21) Appl. No.: 15/499,348

(22) Filed: Apr. 27, 2017

(30) Foreign Application Priority Data
Apr. 27, 2016 (DE) 102016207156.4

(51) Int. Cl. G01R 33/54; A61B 5/00; A61B 5/055; G06F 19/00

(52) U.S. Cl. CPC G01R 33/54; G06F 19/3406; G06F 19/322; A61B 5/0555; G01R 33/543; A61B 5/742; A61B 2576/00

(57) ABSTRACT

An operating method for a medical imaging apparatus includes an imaging program stored in a memory, with which parameters for controlling image data generation will be predetermined. The imaging program has a number of routine examination steps. At least one variant, which is likewise stored in the memory and which is able to be selected by a user instead of the routine examination step for image data generation, exists at least for one of the routine examination steps.

### BACKGROUND OF THE INVENTION

### Field of the Invention

The present invention relates to an operating method for a medical imaging apparatus with an imaging program stored in a memory, with which parameters for controlling generation of imaging data will be predetermined, wherein the imaging program includes a number of routine examination steps.

### Description of the Prior Art

A medical imaging apparatus is an apparatus for acquiring, processing, evaluating and/or storing imaging information in the form of image data. Acoustic methods such as ultrasound (US), emission methods such as Emission Computed Tomography (ECT) and Positron Emission Tomography (PET), optical methods, radiological methods such as x-ray tomography and computed Tomography (CT) can be used to acquire the imaging information for example. The acquisition can also be done by Magnetic Resonance Tomography (MR or MRT) or by combined methods. The medical imaging apparatus can deliver 2-dimensional (2D) or multidimensional, such as 3-dimensional (3D) or 4-dimensional (4D) image data (3 spatial dimensions, resolved over time), which can preferably be stored and/or processed in various formats. The medical imaging apparatus can be used in diagnostics, for example in medical diagnostics.

The imaging of the medical imaging apparatus will be influenced by a number of parameters. Depending on the region of the body, the imaging characteristic of the region, the time available for the imaging, the medical issue etc., there are appropriate imaging programs with routine examination steps, which have proved in practice to be suitable for imaging. The corresponding routine examination steps will be defined by specific values of the parameters influencing the imaging. The totality of the values of the parameters of a routine examination step can be stored as a respective parameter set in a database, so that these can be used again. With imaging by magnetic resonance in particular, a parameter set for the imaging of a region of the body for a specific medical issue can involve several hundred individual parameters, some of which can influence each other to a considerable extent.

During operation of the medical imaging apparatus, that routine examination steps will be changed or optimized for a particular medical issue, or for improved imaging. Conventionally, the changes of the parameters have been documented by individual notes of the users. Improvements memorialized in this way are consequently poorly documented and often barely still comprehensible for the respective user himself or herself after some time. Such changes to the routine examination steps are generally not accessible at all and/or often also not comprehensible to other users.

### SUMMARY OF THE INVENTION

An object of the present invention is to provide an operating method for a medical imaging apparatus, with which changes to parameters for the imaging can be made reproducibly, efficiently, in a coordinated manner and comprehensibly.

The inventive operating method is based on an imaging program stored in a memory, with which parameters for controlling generation of imaging data will be predetermined, wherein the imaging program has a number of routine examination steps. In accordance with the invention at least one variant exists for one of the routine examination steps, which is likewise stored in the memory and which is able to be selected by a user instead of the routine examination step for image data generation.

Through the provision of a variant of a specific routine examination step by the operating method, each operator or user of the imaging apparatus will generally be provided with the option of using this variant for imaging. Particularly with many operators or users, the variant is also available to operators who are not originators of the changes and adaptations. This makes available a defined process for changing and improving imaging sequences. In the event of problems with using the variant the original routine examination step can easily be selected once again and activated for imaging. Thus in parallel to imaging with routine examination steps that are released for imaging, work can be done on variants with the aim of making an improvement. One focus of the invention lies in understanding the improvement of an examination step as a process with operating steps and supporting this process explicitly through the operating system. Previously the focus of the operating system lay with the examination steps themselves and not with changes and improvements to them.

In an embodiment, the variant is assigned a validation code. The extent to which the variant fulfills practical requirements can be specified by means of the validation code. Thus the level of development or the status of the variant is visible for each user.

In another embodiment, each routine examination step and each variant thereof is assigned an identification code to distinguish it. The identification code is assigned automatically, as soon as a variant is created. Via the identification code the operating method automatically recognizes all variants existing for a routine examination step and can offer these for selection quickly and efficiently.

In a further embodiment, the variant is able to be created by a user by changing the routine examination step. A variant of a routine examination step can in this way be developed easily by making corresponding changes to the routine examination step. The creation of variants, which are intended to produce an improvement or an optimization of the corresponding routine examination step, is thus also less prone to errors, since its starting point is a basically already practically-proven routine examination step. It is not necessary to develop the variant as a completely new variant.

In a further embodiment, the variant is able to be modified. In this way a variant can be further developed until it is mature enough to be used in practice. The development stage can be specified by a corresponding setting of the validation code. After the variant has been used for imaging to test it out the validation code can also be changed in accordance with the imaging result.

In another embodiment, the operating method is especially advantageously suited to the operation of a magnetic resonance apparatus. In magnetic resonance imaging, as mentioned, in some cases hundreds of parameter values are to be set, which can often influence each other to a significant extent.

### BRIEF DESCRIPTION OF THE DRAWINGS

FIG. 1 shows an overview of a diagnostic magnetic resonance device, for imaging a region of a body, with a control console and a monitor.

FIG. 2 shows the structure of an imaging program with validation code, identification code and comments field.

FIG. 3 shows a diagram of a selection option on the monitor of the control console for use of routine examination steps or variants thereof.

FIG. 4 shows a diagram of the development status of a variant.

FIG. 5 shows a diagram of a selection option for use of the variant at the beginning of imaging.

### DESCRIPTION OF THE PREFERRED EMBODIMENTS

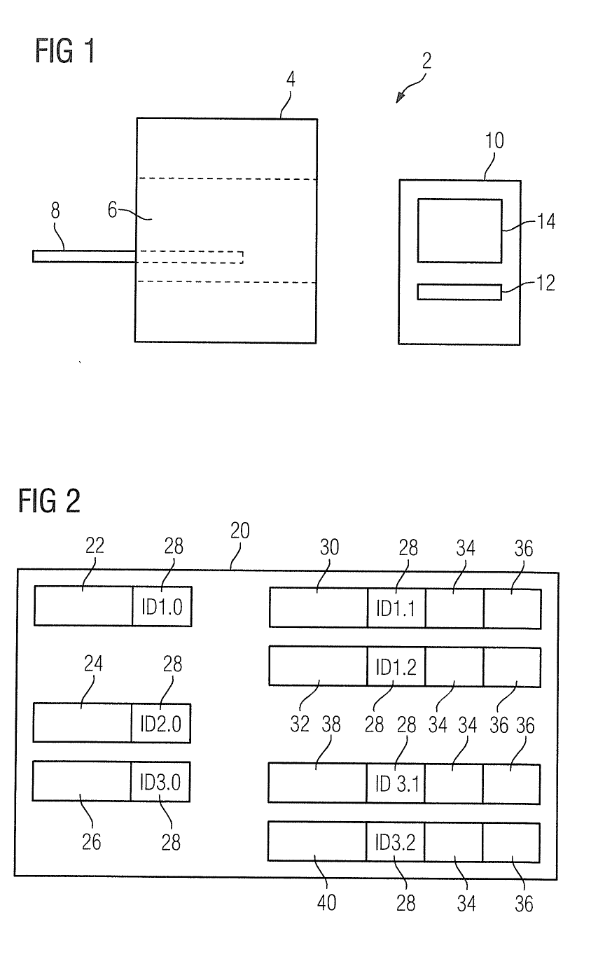

FIG. 1 shows a diagnostic magnetic resonance (MR) apparatus 2 with an MR data acquisition scanner 4. The scanner has a tunnel-shaped inner space 6, in which a patient supported on a patient couch 8 will be positioned for imaging. The scanner contains, as is known, gradient coils for spatially encoding MR signals and radio-frequency coils for excitation of nuclear spins and for receipt of the resulting MR signals, as well as further hardware components. The scanner 4 is controlled by a control computer 10. The control computer 10 includes a system processor and an image processor with associated data memories, as well as a user interface, which is connected to the system processor and the image processor, and control software. The control computer 10 additionally includes a pulse sequence controller and a gradient pulse shape generator. The user interface includes an input unit 12 and a monitor 14. The positioning of the patient and the operation of the gradient and radio-frequency coils will be predetermined by an imaging program executed by the system processor together with the pulse sequence controller and the gradient pulse shape generator.

For many regions of the body and clinical issues, there are correspondingly adapted imaging programs. For example, there are a number of different imaging programs for the head region, such as for general examinations without a specific medical focus. Furthermore for the head region, there are likewise specific imaging programs for lesions, for injuries, for neurological degeneration, for epilepsy, etc. All these imaging programs are stored in a database and can be retrieved from the database for imaging. The respective imaging program generally includes a number of examination steps. The examination steps differ, for example, in the pulse sequences used and/or in different imaging characteristics for the different body tissue and body fluids. In addition the examination steps include values for the parameters of the pulse sequences that can be set. For example, the examination steps contain pulse sequences with T1 or T2 weighting, the creation of 2D or 3D image data, etc. Parameters that can be set are, for example, the spatial location of the image data in relation to the patient being examined, such as transverse or coronal, the slice thickness, the resolution, etc. Examination steps that have been proven in practice and in clinical routines will be referred to below as routine examination steps.

Normally these routine examination steps meet the demands made on imaging. However in specific application cases attempts will be made by users to further improve the imaging for a particular diagnostic issue. To do this, the user takes an available routine examination step and changes one or more parameter settings with the aim of improved imaging. However, known operating methods for medical imaging apparatuses do not support variants created in this way from already-established routine examination steps.

FIG. 2 shows, in an exemplary embodiment of the invention, a program and data structure, with which changes or variants of routine examination steps will be supported by the operating method. According to this embodiment, an imaging program 20 has a number of routine examination steps 22, 24, 26, which are each provided with a unique identification code 28. In the exemplary embodiment, the value of the identification code 28 for routine examination step 22 is ID1.0, for routine examination step 24 is ID2.0, and for routine examination step 26 is ID3.0. In FIG. 2 only three routine examination steps 22, 24, 26 are shown as an example, but in reality imaging programs could include significantly more routine examination steps.

For routine examination step 22, there are two variants 30, 32 in which, in each case, by comparison with routine examination step 22, a few parameters have been changed with the aim of making the imaging more informative. Each of the two variants 30, 32 is likewise assigned a unique identification code 28. The identification code 28 for the variant 30 has the value ID1.1 and the identification code 28 for the variant 32 has the value ID1.2. The data structure of the identification code 28 makes it easy to identify the routine examination step to which the respective variant belongs. The first digit in the identification code 28 specifies the associated routine examination step, the second digit of the identification code 28 (after the period) individualizes the variants assigned to the respective routine examination step.

Furthermore the variants 30, 32 are assigned a validation code 34 and a text field 36 as a comments field in each case. By contrast with the identification code 28, the values of the validation code 34 and the contents of the comments field 36 can be changed by an operator or user. The values of the validation code 34 are intended to specify the development and maturity status of the respective variant and can for example for reasons of systemization only assume fixed predetermined values. With, for example, four permissible values of the validation code, 34 the values can stand for “work in progress”, “validation”, “released” and “outdated”. Then the value “work in progress” is intended to mean that the variant has just been created and is being worked on, it is not yet suitable for imaging. The value “validation” is intended to mean that the variant can be used for trials and assessment. The value “released” is intended to mean that the variant can be used for everyday imaging. Finally the value “outdated” is intended to mean that there is a better version for the original routine examination step and that the “outdated” version is no longer recommended for use.

As a further example, it is also shown in FIG. 2 that there are likewise variants 38, 40 for the routine examination step 26. The values of the identification code 28 are defined in relation to the routine examination step 26 and the corresponding variants 38, 40. Accordingly, as is described above, further routine examination steps can likewise be assigned variants.

The user starts an improvement process of a routine examination step 22, 26, by selecting the corresponding routine examination step 22 or 26 and marking it and storing it as variant 30 or 32 or 38 or 40. Each stored new variant is given the validation code 34 “work in progress” by the operating system and an identification code 28 in accordance with the pattern described above. This variant can then be changed and after a preliminary conclusion to the changes and improvements, can be set to the status “VERIFY”. The variant is then free to be tried out in practice.

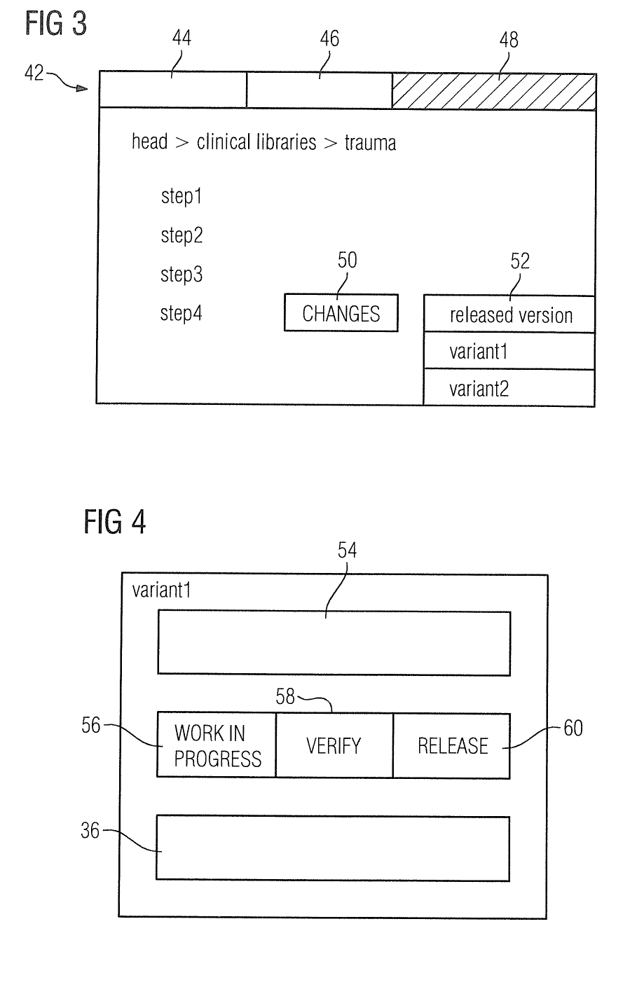

FIG. 3 shows an example of an input and selection mask to be displayed on the monitor 14, via which the routine examination steps 22, 24, 26 or a variant 30, 32, 38, 40 thereof are displayed and selected. In a header area 42 of the input and selection mask, buttons 44, 46, 48 are provided, via which different functionalities of the input and selection mask for a selected imaging program are able to be controlled. Here the imaging program “trauma” for the region “head” in the subdirectory “clinical libraries” is selected. Thus the activation of the button 44 allows editing of the imaging program 20, the button 46 allows a simulation of the imaging program 20 and the button 48 principally allows the administration of the imaging program 20 including the individual examination steps for this program. In the example of FIG. 3, the button 48 is activated, which is marked or symbolized by cross-hatching. With this button the user is shown a list of the routine examination steps used during imaging, here “step1” to “step4”. In addition, with a button 50 and a warning “CHANGES”, it is indicated that one or more variants exist for routine examination step “step4”. By activating the button 50 the changes and explanations for them are displayed, as will be further explained later with reference to FIG. 4. In addition further buttons 52 will be activated for actuation, which are provided as selection buttons for the routine examination step to be used or also variants thereto. In the example the user can use a proven and released version of the routine examination step “step4” or also one of the variants “variant1” or “variant2” for imaging.

In FIG. 4 a user interface is shown as an example, which opens when the button 50 (see FIG. 3) has been activated. Displayed in a text field 54 is a brief description of the corresponding variant, here the variant “variant1”. Arranged below the text field 54 are buttons 56, 58, 60 for a corresponding activation of the validation code. Via the activation of one of the buttons 56, 58, 60 by the user the corresponding development state of the variant “variant1” specified there can be entered or changed. After the activation of the corresponding button 56, 58, 60, said button will be shown highlighted. For example “WORK IN PROGRESS” can be specified for the variant “variant1” via the button 56, meaning that the variant is at the development stage and that work is still being done on it. The variant “variant1” is not yet to be used for imaging in this state. A button 58, shown highlighted, signals to the user with the advice “VERIFY”, that this variant is available for testing and that comments on the function and quality of imaging when it is used are desired. In the “VERIFY” status the comments field 36 is likewise opened for entry of feedback about the function and quality of the variant. After successful tests the variant can be made available for general imaging for all users by means of activation of the button 60 “RELEASE”. At the same time the previously used routine examination step can be provided with the advice “OUTDATED”, because an improved variant for it exists.

Via an actuation of the buttons 56, 58, 60 the user can change the functionality of the operating system with regard to content and thus also set the marker or validation code at the corresponding button. Through this action, the procedure that will be used subsequently for the patient will be directly influenced to some extent. If, for example, the marker or validation code will be set to “RELEASE” for “variant1”, this variant will be used as from the next patient. If, however, a new variant is created and this is still in the state “WORK IN PROGRESS”, this will be ignored at least as a start value or default for a patient, since normally only released variants will be used.

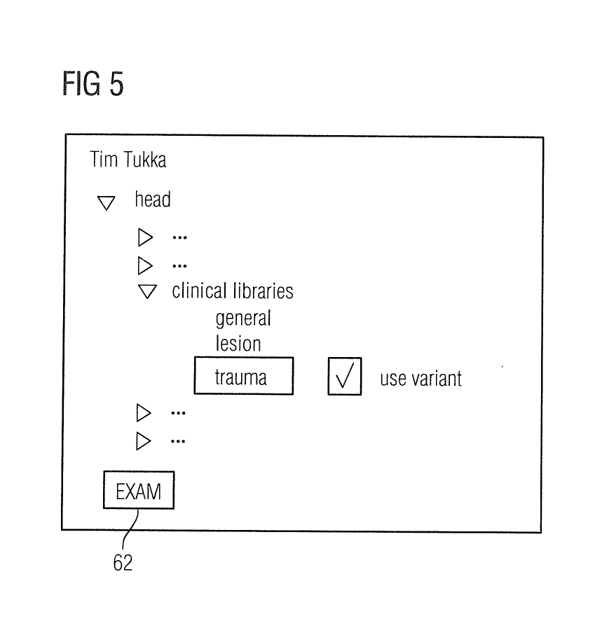

FIG. 5 shows a user interface as an example for a workflow of an imaging controlled by the operating system, as can be displayed during a patient registration at the beginning of an imaging. In this case the user, after entering the patient name, here for example “Tim Tukka”, has opened the examination region “head”. From the plurality of possible examinations the user has then opened the menu entry “clinical libraries” and selected the imaging program “trauma” there. For the imaging program “trauma”, in addition to a routine examination step contained therein, there is a variant, which can be activated for imaging by checking a box. After the selection of the imaging program the imaging will be started by activating the button 62.

In an alternative to the selection option right at the beginning of an imaging in accordance with FIG. 5, after conclusion of an imaging with an imaging program with routine examination steps, which are recommended for use or released, there can also be a reference to variants in the current imaging program. The validation code “work in progress” or “validation” can be used for this purpose for example. The operating system now offers the opportunity of inserting one or more not-yet-released program steps instead of the released routine examination steps. This is worth doing if the user still has some time. The new imaging can be started via a button with the variant or the variants. After conclusion of the new imaging, feedback on the function and quality of the variant will be requested. This enables feedback to be asked for systematically.

Each use of a variant in status “VERIFY” for imaging can be stored as additional information for documentation purposes and be subjected to a quality check.

In summary the following advantages are produced. A defined process will be predetermined by the operating system for the improvement and changing of examination steps, which have initially generally been proven in practice. The operating system supports the changes and development stages and also displays the current development stages. Likewise the exchange of alternatives and variants for existing routine examination steps will be supported by the operating system. This enables errors to be avoided. Changes and the existence of variants are transparent and visible for all users. The functionality and quality of the changed examination steps can be systematically surveyed via a feedback option. In the case of problems during the imaging with a variant the operating system supports the option of referring back to older alternatives. Finally the complete change process is documented by the operating system. The changes are verifiable for all users. The operating system described here is especially highly efficient when a large number of medical imaging apparatuses are to be maintained and updated centrally and to some extent in an automated manner. Here an improved examination step not only acts on an imaging apparatus, but on many imaging apparatuses. A systematic process for making improvements to the imaging is essential here.

Although modifications and changes may be suggested by those skilled in the art, it is the intention of the Applicant to embody within the patent warranted hereon all changes and modifications as reasonably and properly come within the scope of the Applicant's contribution to the art.

1. An operating method for a medical imaging apparatus comprising:
from a processor, accessing an imaging program, for operating a medical imaging apparatus, that is stored in a memory, the imaging program stored in the memory comprising a plurality of routine examination steps in which parameters for controlling generation of image data with said medical imaging apparatus are predetermined;
also from said processor, accessing at least one variant for at least one of said routine examination steps from said memory in response to a user input into said processor;
presenting the accessed at least one variant for at least one of said routine examination steps at a display in communication with said processor and, via said processor, accepting a user input that causes said at least one variant of said at least one routine examination step to the selected and inserted into said imaging program in place of the routine examination step with said predetermined parameters; and
from said processor, emitting an electronic signal, representing said imaging program with the selected variant of said at least one routine examination step, in a form configured to operate said medical imaging apparatus.

2. A method as claimed in claim 1 comprising storing said variant in said memory with a validation code assigned thereto.

3. A method as claimed in claim 2 comprising allowing a user, via said processor, to change said validation code.

4. A method as claimed in claim 1 comprising, in said memory, assigning said at least one routine examination step and said variant and identification code that distinguishes between said at least one routine examination step and said variant.

5. A method as claimed in claim 1 comprising creating said variant, via said processor, by changing a routine examination step by user interaction.

6. A method as claimed in claim 1 comprising allowing said variant to be changed via said processor.

7. A method as claimed in claim 1 comprising providing said variant with a text field in which comments can be entered via said processor.

8. A method as claimed in claim 1 comprising selecting said variant during patient registration prior to operation of said medical imaging apparatus for said patient.

9. A method as claimed in claim 1 comprising allowing selection of said variant at an end of an imaging session employing said routine examination step.

10. A method as claimed in claim 1 comprising logging a number of uses of said variant in said memory.

11. A method as claimed in claim 1 wherein said medical imaging apparatus is a magnetic resonance apparatus.
